(19) United States
(12) Patent Application Publication
(10) Pub. No.: US 2020/0088727 A1
(43) Pub. Date: Mar. 19, 2020

(54) HUMAN PAPILLOMA VIRUS DETECTION

(71) Applicant: X Squared Health LLC, Durham, NC (US)

(72) Inventors: Megan Harrison Dew, Durham, NC (US); Jewell Brey, Chapel Hill, NC (US); Jazmin McCorkle, Sherrills Ford, NC (US); Andrea Morgan Rich, Asheboro, NC (US); Melissa Serrano Burns, Pataskala, OH (US)

(73) Assignee: X Squared Health LLC, Durham, NC (US)

(21) Appl. No.: 16/135,732

(22) Filed: Sep. 19, 2018

(51) Int. Cl. G01N 33/569; G01N 33/52; G01N 33/493; C07K 16/08; C07K 16/32

(52) U.S. Cl. CPC G01N 33/56983; G01N 33/525; C07K 16/32; C07K 16/084; G01N 33/493

(57) ABSTRACT

The invention is a self-administered urine based lateral flow immunoassay that detects the presence of an HPV oncogene and thus the presence in a test subject of HPV.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to the detection of Human Papilloma Virus or HPV. In particular, it relates to a home urine test which can identify the presence of HPV in the urine.

### Description of Related Art

The detection of cervical cancer in populations is frequently resource limited. This is especially true in populations in underdeveloped countries. It has been known for some time that HPV is the obligate cause of cervical cancer. A number of tests have been developed for detection of HPV including Pap testing and protein detection.

The serious drawbacks of the present testing methods include the long delay in getting results and the need to involve a clinician to administer the test. Even the best of the tests, e.g. Hybrid Capture 2, needs to be run by someone with training. The vast majority of the world population cannot afford or does not have access to testing necessary to detect HPV. Home type testing, i.e. testing done by the user alone, is not currently available and thus testing is unavailable to most of the world's population to confirm the presence of HPV.

A number of proteins from HPV are known that are associated with a type of cervical cancer. The testing for these proteins has not progressed further from the same type of HPV testing as other methods. It is not clear then that any method of home testing for HPV is viable or even available.

### BRIEF SUMMARY OF THE INVENTION

The present invention relates to the discovery of a method of testing for HPV in a home setting. A flow assay involving placing urine on a test strip impregnated with antibodies to one or more HPV proteins which reports a color change on binding to the proteins is utilized on a home test strip kind of home use device.

Accordingly, in one embodiment, there is a home HPV test strip comprising one or more antibodies to one or more proteins of HPV wherein there is a visible color change resulting from the binding of the one or more HPV proteins to the one or more antibodies derived from urine placed on the test strip with the one or more antibodies on the test strip.

Accordingly, in another embodiment, there is a method of testing for the presence of HPV in a human subject comprising:
a) taking a urine sample from the human subject;
b) placing the urine on a test strip having one or more antibodies to one or more HPV proteins wherein the antibodies are associated with a color change agent which is activated by the presence of the a complex of the one or more antibodies or the one or more proteins; and
c) observing the test strip for the presence of a color change indicating the presence of HPV.

### BRIEF DESCRIPTION OF THE DRAWINGS

FIG. 1 is drawing of the initial stage of assay.

FIG. 2 is view of the device in mid-process assay.

FIG. 3 is a view of the device with a completed test assay.

FIG. 4 is a flow chart of the method of using the device in the invention.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “test strip” refers to a strip of the kind used for the purpose of placing urine on a particular spot which initiates a color change in the presence of a protein of HPV. These tests can be sticks, strips, or the like, and the basic strip construction is well known. They generally utilize flow of urine to the antibody utilizing a membrane to pass urine through. In one embodiment of the test strip, a lateral flow construction is utilized. In a lateral flow assay, the urine sample flows toward a wicking material by capillary action from its application point, past the one or more antibodies wherein the antibodies bind to one or more proteins in the sample until they reach a test line where the color change occurs giving a visible indication of a positive test result from binding to the protein antibody complex. Frequently, this is accomplished as line of color. In addition, a control test can be included such as including an anti-IgG antibody. By home test is meant that it is capable of being used by someone at home without need of a third party.

As used herein, the term “proteins” refers to proteins produced by a cell in response to HPV. In one embodiment, it is specific to proteins which are unique to HPV response. In another embodiment, “proteins” refers to one or more HPV response proteins. In one embodiment, the proteins are the HPV L1, L2, and E6.

As used herein, the term “antibodies to proteins” refers to one or more antibodies (mono or polyclonal) that binds a protein produced in reaction to HPV such as L1, L2, and E6. Since antibodies can be various sizes and attached at different portions of the protein, the inclusion of different antibodies to the same protein is also contemplated. In one embodiment, the antibodies are conjugated with gold nanoparticles to illicit a color change in the presence of HPV.

As used herein, the term “color change” refers to a color changing chemical marker indicator. It is usually a label that when a binding of the antibodies occurs, it causes a visible color change on the test strip. Labels include enzymes, dyes and chemiluminescence agents, and the like and are known within the industry.

As used herein, the term “HPV test” refers to a test for the detection of one or more HPV protein(s) present in the urine of a test subject to aid in the early detection of HPV, and ultimate prevention of cervical cancer which may be associated with the presence of HPV. A positive on the test illustrated by the color change of the test strip when the HPV protein conjugated to an antibody is detected indicates the presence of HPV and results in a color change (on the test strip when the protein conjugated to an antibody is detected), indicating the presence of HPV.

As used herein, the term “concentration device” refers to a device which concentrates the proteins in the urine sample, e.g. using a filter membrane or other concentration device or method during the process.

### DRAWINGS

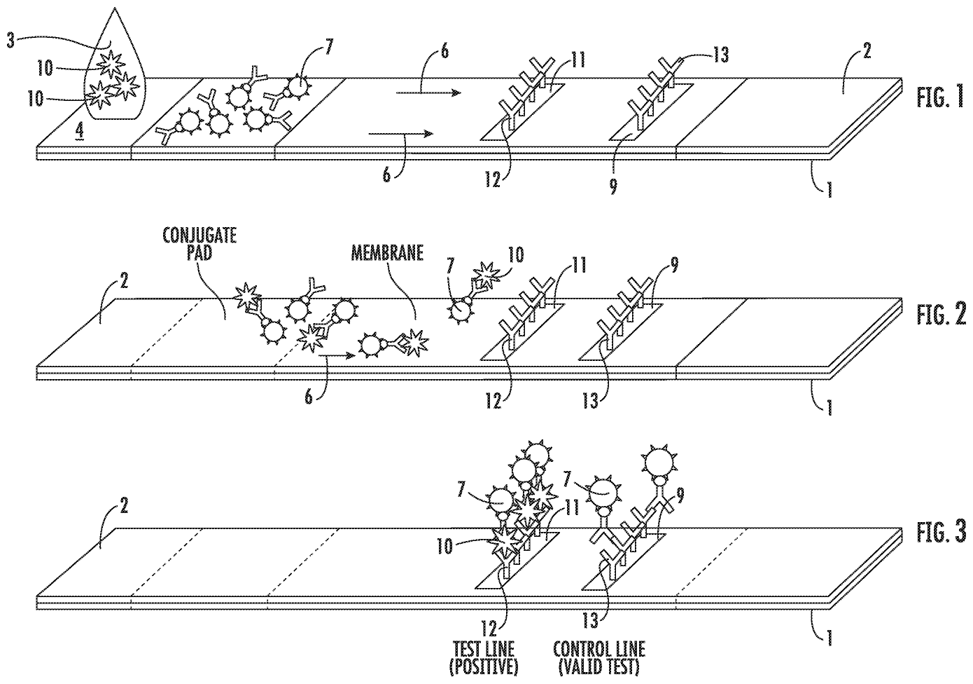

Now referring to the drawings, FIG. 1 shows the initial start of a test using the test strip of the invention. A test strip 1 having a wicking pad for lateral flow 2 has an initial urine sample 3 containing HPV proteins 10 placed on the start end 4 of test strip wicking pad 2. Capillary flow 6 causes the sample to move toward antibodies 7 (being the same or different as described herein) placed on the test pad. They continue moving toward test line 11 which also has one or more antibodies 12 to the protein. In this embodiment, the antibodies are conjugated with gold nanoparticles. A control line 9 is shown as well which is composed of another antibody 13 designed to bind with the free antibodies 7 designed as a control.

FIG. 2 shows the test strip 1 where the urine sample 3 has flowed past the antibodies 7 and proteins 10 are attached to the antibodies 7. The antibody oncogene complex is moving toward the test line 9. In FIG. 3, the test is shown as a completed positive test having detected the protein antibody complex. Antibodies 7 with attached protein 10 have bound to the fixed antibody 12 on the test line. The binding causes a color change indicating a positive test. At the same time, the control line will bind the free antibodies, molecules, particles or the like indicating a valid test.

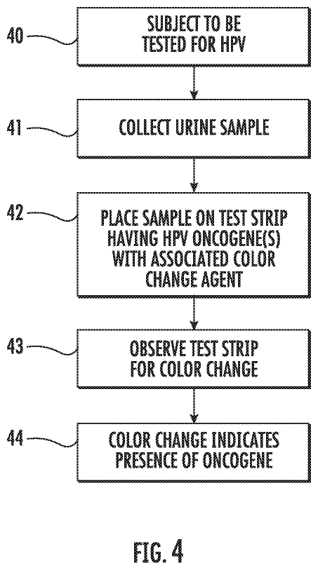

FIG. 4 is a flow chart of the method of the present invention. In this method, a subject to be tested for HPV 40 is selected. A urine sample is then collected 41 from the subject. Next, the sample is placed on the test strip 42 wherein the test strip has a HPV anitbody(ies) with an associated color change agent. Observation of the test strip 43 for a color change is then done wherein a color change indicates the presence of the protein 44 and thus the presence of HPV.

### Experimental

In order to determine if an at-home HPV detection device would be wanted and needed by women, an anonymous survey was conducted. Five-hundred women, primarily United States citizens age 18-60 were surveyed and asked the following question: if there was a non-invasive at-home test that could determine if you are infected with HPV, would you take it? The results showed that 91% of women would be willing to take the test; this number increased to 95% when women were presented with facts regarding the relationship between HPV and cervical cancer. This indicates that a test of this nature is both needed and wanted by women.

As a proof of concept, an ELISA (enzyme linked immunosorbent assay) test was performed. The purpose of this test was twofold: to determine if we could detect the E6, L1, and L2 protein reliably in laboratory settings using a test that performs analogously to a lateral flow test, and to determine the minimum concentration of protein that could be detected in this way. A commercial sandwich ELISA kit that binds the E6 to two different antibodies was used to perform the test. The kit contained a 96 well plate coated with capture antibodies, a second detection antibody, an enzyme that binds to the second antibody, a substrate that reacts with the enzyme to produce a color change, and all necessary buffers and reagents. The assay was set-up and performed in accordance with the instructions of the kit, with the included E6 protein diluted to various levels.

To determine the concentration of protein captured based on color, the absorbance was measured with a microplate reader set to 450 nm. The results of the test verified that the protein can be bound by antibodies and used in a lateral flow test. It was further determined that the standard E6 protein supplied with the kit was able to be detected accurately down to a concentration of 0.313 ng/mL. This tells us that for a lab setting, we need the urine samples to have E6 protein concentration of at least 0.313 ng/mL to be detected with an ELISA assay. This concentration may change when we transition to a lateral flow test strip but that is unknown at this time.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A home HPV lateral flow assay test strip comprising a start end, a middle, and an end point having one or more HPV antibodies positioned in a middle, a position for placement of a urine sample at the first end, and a color change system at an end point for indicating when the urine containing the one or more proteins of HPV has been wicked to the middle and binds to the one or more HPV antibodies which is wicked to the end point where it reacts with the color change system to produce an indicator that there is an HPV protein in the sample.

2. The test according to claim 1 wherein the protein antibodies are one or more antibodies to at least one of L1, L2, and E6.

3. The test according to claim 2 wherein there are at least two antibodies.

4. The test according to claim 1 wherein one of the antibodies is E6.

5. The test according to claim 1 wherein the color change is due to a chemical marker bound or associated with the one or more antibodies in the lateral flow assay test strip.

6. The test according to claim 1 wherein the color change is from an enzyme, dye, gold nonparticle, or chemiluminescence agent associated with the binding of an oncogene.

7. The test according to claim 2 wherein each antibody is associated with a different color when binding the protein.

8. The test according to claim 2 wherein there are two or more antibodies for the same protein.

9. (canceled)

10. The test according to claim 1 wherein there are antibodies to produce a control result on the lateral flow assay test strip.

11. The test according to claim 1 wherein there is a device for concentrating the urine.

12. The test according to claim 11 wherein the concentrating device is a filter membrane.

13. A method of testing for the presence of HPV in a human subject comprising:
a) taking a urine sample from the human subject;
b) placing the urine on a lateral flow assay test strip first end, having one or more antibodies to one or more HPV proteins in a middle of the lateral flow assay test strip and a color change agent at an end point on the lateral flow assay test strip which is activated by the presence of a complex of the one or more antibodies and the one or more proteins; and
c) observing the lateral flow assay test strip for the presence of a color change indicating the presence of HPV.
